(19) United States
(12) Patent Application Publication
(10) Pub. No.: US 2002/0004218 A1
(43) Pub. Date: Jan. 10, 2002

(54) Methods for identifying compounds useful for inhibiting geranylgeranyl diphosphate synthase

(76) Inventors: Gideon A. Rodan, Bryn Mawr, PA (US); Alfred A. Reszka, Glenside, PA (US)

Correspondence Address: MERCK AND CO INC, P O BOX 2000, RAHWAY, NJ, 070650907

(21) Appl. No.: 09/817,432

(22) Filed: Mar. 26, 2001

Related U.S. Application Data
(60) Provisional application No. 60/194,263, filed on Mar. 31, 2000.

(51) Int. Cl. C12Q 1/42

(52) U.S. Cl. 435/21

(57) ABSTRACT

The present invention relates to methods for identifying compounds useful as inhibitors of geranylgeranyl diphosphate synthase. More particularly, the compounds so identified are useful for inhibiting bone resorption. The present invention also relates to methods for inhibiting bone resorption in a mammal comprising administering to a mammal in need thereof a therapeutically effective amount of a geranylgeranyl diphosphate synthase inhibitor.

### BRIEF DESCRIPTION OF THE INVENTION

The present invention relates to methods for identifying compounds useful as inhibitors of geranylgeranyl diphosphate synthase. More particularly, the compounds so identified are useful for inhibiting bone resorption. The present invention also relates to methods for inhibiting bone resorption in a mammal comprising administering to a mammal in need thereof a therapeutically effective amount of a geranylgeranyl diphosphate synthase inhibitor.

### BACKGROUND OF THE INVENTION

A variety of disorders in humans and other mammals involve or are associated with abnormal bone resorption. Such disorders include, but are not limited to, osteoporosis, glucocorticoid induced osteoporosis, Paget's disease, abnormally increased bone turnover, periodontal disease, arthritis, osteoarthritis, rheumatoid arthritis, tooth loss, bone fractures, rheumatoid arthritis, periprosthetic osteolysis, osteogenesis imperfecta, metastatic bone disease, hypercalcemia of malignancy, and multiple myeloma. One of the most common of these disorders is osteoporosis, which in its most frequent manifestation occurs in postmenopausal women. Osteoporosis is a systemic skeletal disease characterized by a low bone mass and microarchitectural deterioration of bone tissue, with a consequent increase in bone fragility and susceptibility to fracture. Osteoporotic fractures are a major cause of morbidity and mortality in the elderly population. As many as 50% of women and a third of men will experience an osteoporotic fracture. A large segment of the older population already has low bone density and a high risk of fractures. There is a significant need to both prevent and treat osteoporosis and other conditions associated with bone resorption. Because osteoporosis, as well as other disorders associated with bone loss, are generally chronic conditions, it is believed that appropriate therapy will typically require chronic treatment.

Normal bone physiology involves a process wherein bone tissue is continuously being turned over by the processes of modeling and remodeling. In other words, there is normally an appropriate balance between resorption of existing bone tissue and the formation of new bone tissue. The exact mechanism underlying the coupling between bone resorption and formation is still unknown. However, an imbalance in these processes is manifested in various disease states and conditions of the skeleton.

Two different types of cells called osteoblasts and osteoclasts are involved in the bone formation and resorption processes, respectively. See H. Fleisch, Bisphosphonates In Bone Disease, From The Laboratory To The Patient, 3rd Edition, Parthenon Publishing (1997), which is incorporated by reference herein in its entirety.

Osteoblasts are cells that are located on the bone surface. These cells secrete an osseous organic matrix, which then calcifies. Substances such as fluoride, parathyroid hormone, and certain cytokines such as protaglandins are known to provide a stimulatory effect on osetoblast cells. However, an aim of current research is to develop therapeutic agents that will selectively increase or stimulate the bone formation activity of the osteoblasts.

Osteoclasts are usually large multinucleated cells that are situated either on the surface of the cortical or trabecular bone or within the cortical bone. The osteoclasts resorb bone in a closed, sealed-off microenvironment located between the cell and the bone. The recruitment and activity of osteoclasts is known to be influenced by a series of cytokines and hormones. It is well known that bisphosphonates are selective inhibitors of osteoclastic bone resorption, making these compounds important therapeutic agents in the treatment or prevention of a variety of systemic or localized bone disorders caused by or associated with abnormal bone resorption. However, despite the utility of bisphosphonates, there remains the desire amongst researchers to develop additional therapeutic agents for inhibiting the bone resorption activity of osteoclasts.

The mevalonate biosynthetic pathway is an important pathway of osteoclast function. This pathway is involved in the bisosynthesis of cholesterol and of isoprenoids, some of which are used in protein prenylation. The enzyme geranylgeranyl disphosphate synthase (GGPP synthase) mediates the synthesis of geranylgeranyl diphosphate by catalyzing the condensation of one molecules of farnesyl diphosphate (FPP) with one molecule of isopentenyl diphosphate (IPP) to form geranylgeranyl diphosphate (GGPP), or alternatively, at a slower rate in vitro, the sequential condensation of three molecules of isopentenyl diphosphate (IPP) and one molecule of dimethylallyl diphosphate (DMAPP) to produce geranylgeranyl diphosphate (GPP).

Geranylgeranyl diphosphate is essential for the geranylgeranylation of several proteins required for cytoskeletal organization and vesicular traffic control. Interference with the function of these proteins can also lead to apoptosis, i.e. programmed cell death. Therefore, geranylgeranyl diphosphate synthase, the enzyme involved in the synthesis of geranylgeranyl diphosphate, is essential for the proper biological functioning of the osteoclasts.

It would be highly desirable to identify and develop compounds useful as selective inhibitors of geranylgeranyl diphosphate synthase in the osteoclasts. Such inhibitors would be useful for inhibiting ostetoclast function, thereby inhibiting undesired bone resorption and its manifestations.

In the present invention it is surprising found that nitrogen-containining bisphosphonates such as alendronate and risedronate are specific nanomolar inhibitors of geranylgeranyl diphosphate synthase. It is also surprisingly found that it is possible to identify other compounds useful as geranylgeranyl disphosphate synthase inhibitors.

In the present invention it is also found that inhibitors of geranylgeranyl diphosphate synthase are useful for inhibiting bone resorption. Without being limited by theory, it is believed that these inhibitors are responsible for inhibiting the bone resorption activity of the osteoclasts.

It is an object of the present invention to provide methods for identifying compounds useful as geranylgeranyl diphosphate synthase inhibitors.

It is an object of the present invention to provide methods for inhibiting geranylgeranyl diphosphate synthase in a mammal comprising administering to a mammal in need thereof a therapeutically effective amount of a geranylgeranyl disphosphate synthase inhibitor having an IC₅₀ value from about 0.01 nanoM to about 1000 nanoM.

It is an object of the present invention to provide methods for inhibiting bone resorption in a mammal comprising administering to a mammal in need thereof a therapeutically effective amount of a geranylgeranyl disphosphate synthase inhibitor having an IC₅₀ value from about 0.01 nanoM to about 1000 nanoM.

It is another object of the present invention to provide methods for treating or reducing the risk of contracting a disease state or condition mediated by famesyl disphosphate synthase in a mammal comprising administering to a mammal in need thereof a therapeutically effective amount of a geranylgeranyl disphosphate synthase inhibitor having an IC₅₀ value from about 0.01 nanoM to about 1000 nanoM.

It is another object of the present invention to provide methods for treating or reducing the risk of contracting a disease state or condition involving or affecting bone tissue in a mammal comprising administering to a mammal in need thereof a therapeutically effective amount of a geranylgeranyl disphosphate synthase inhibitor having an IC₅₀ value from about 0.01 nanoM to about 1000 nanoM.

It is an object of the present invention to provide methods for inhibiting geranylgeranyl diphosphate synthase activity in a mammal comprising administering to a mammal in need thereof comprising administering to a mammal in need thereof a therapeutically effective amount of the combination of: (a) a geranylgeranyl disphosphate synthase inhibitor having an IC₅₀ value from about 0.01 nanoM to about 1000 nanoM, and (b) a bisphosphonate active.

It is an object of the present invention to provide methods for inhibiting bone resorption in a mammal comprising administering to a mammal in need thereof a therapeutically effective amount of the combination of: (a) a geranylgeranyl disphosphate synthase inhibitor having an IC₅₀ value from about 0.01 nanoM to about 1000 nanoM, and (b) a bisphosphonate active.

It is an object of the present invention to provide methods for treating or reducing the risk of contracting a disease state or condition mediated by geranylgeranyl diphosphate synthase comprising administering to a mammal in need thereof a therapeutically effective amount of the combination of: (a) a geranylgeranyl disphosphate synthase inhibitor having an IC₅₀ value from about 0.01 nanoM to about 1000 nanoM, and (b) a bisphosphonate active.

It is an object of the present invention to provide methods for treating or reducing the risk of contracting a disease state or condition involving or affecting bone tissue in a mammal comprising administering to a mammal in need thereof a therapeutically effective amount of the combination of: (a) a geranylgeranyl disphosphate synthase inhibitor having an IC₅₀ value from about 0.01 nanoM to about 1000 nanoM, and (b) a bisphosphonate active.

It is another object of the present invention to provide pharmaceutical compositions comprising a therapeutically effective amount of a geranylgeranyl disphosphate synthase inhibitor having an IC₅₀ value from about 0.01 nanoM to about 1000 nanoM.

It is another object of the present invention to provide pharmaceutical compositions comprising a therapeutically effective amount of the combination of: (a) a geranylgeranyl disphosphate synthase inhibitor having an IC₅₀ value from about 0.01 nanoM to about 1000 nanoM and (b) a bisphosphonate active.

It is another object of the present invention to provide the use of a composition in the manufacture of a medicament for treating or reducing the risk of contracting a disease state or condition involving bone tissue in a mammal comprising administering to a mammal in need thereof a therapeutically effective amount of a geranylgeranyl diphosphate synthase inhibitor having an IC50 value from about 0.01 nanoM to about 100 0 nanoM.

It is another object of the present invention to provide the use of a composition in the manufacture of a medicament for treating or reducing the risk of contracting a disease state or condition involving bone tissue in a mammal comprising a therapeutically effective amount of a geranylgeranyl diphosphate synthase inhibitor having an IC₅₀ value from about 0.01 nanoM to about 100 0 nanoM.

These and other objects will become readily apparent from the detailed description which follows.

### SUMMARY OF THE INVENTION

The present invention relates to methods for identifying compounds useful as geranylgeranyl diphosphate synthase inhibitors, comprising:

a). contacting a putative geranylgeranyl diphosphate synthase inhibitor with a geranylgeranyl diphosphate synthase solution, and

b). determining the geranylgeranyl diphosphate synthase activity of said solution with a geranylgeranyl diphosphate synthase solution not contacted with said putative inhibitor.

The present invention also relates to methods for inhibiting geranylgeranyl diphosphate synthase in a mammal comprising administering to a mammal in need thereof a therapeutically effective amount of a geranylgeranyl disphosphate synthase inhibitor having an IC₅₀ value from about 0.01 nanoM to about 1000 nanoM.

The present invention also relates to methods for inhibiting bone resorption in a mammal comprising administering to a mammal in need thereof a therapeutically effective amount of a geranylgeranyl disphosphate synthase inhibitor having an IC₅₀ value from about 0.01 nanoM to about 1000 nanoM.

The present invention also relates to methods for treating or reducing the risk of contracting a disease state or condition mediated by geranylgeranyl disphosphate synthase in a mammal comprising administering to a mammal in need thereof a therapeutically effective amount of a geranylgeranyl disphosphate synthase inhibitor having an IC₅₀ value from about 0.01 nanoM to about 1000 nanoM.

The present invention also relates to methods for treating or reducing the risk of contracting a disease state or condition involving or affecting bone tissue in a mammal comprising administering to a mammal in need thereof a therapeutically effective amount of a geranylgeranyl disphosphate synthase inhibitor having an IC₅₀ value from about 0.01 nanoM to about 1000 nanoM.

The present invention also relates to methods for inhibiting geranylgeranyl diphosphate synthase activity in a mammal comprising administering to a mammal in need thereof a therapeutically effective amount of the combination of: (a) a geranylgeranyl disphosphate synthase inhibitor having an IC₅₀ value from about 0.01 nanoM to about 1000 nanoM, and (b) a bisphosphonate active.

The present invention also relates to methods for inhibiting bone resorption in a mammal comprising administering to a mammal in need thereof a therapeutically effective amount of the combination of: (a) a geranylgeranyl disphosphate synthase inhibitor having an IC₅₀ value from about 0.01 nanoM to about 1000 nanoM, and (b) a bisphosphonate active.

The present invention also relates to methods for treating or reducing the risk of contracting a disease state or condition mediated by geranylgeranyl diphosphate synthase comprising administering to a mammal in need thereof a therapeutically effective amount of the combination of: (a) a geranylgeranyl disphosphate synthase inhibitor having an IC₅₀ value from about 0.01 nanoM to about 1000 nanoM, and (b) a bisphosphonate active.

The present invention also relates to methods for treating or reducing the risk of contracting a disease state or condition involving or affecting bone tissue in a mammal comprising administering to a mammal in need thereof a therapeutically effective amount of the combination of: (a) a geranylgeranyl disphosphate synthase inhibitor having an IC₅₀ value from about 0.01 nanoM to about 1000 nanoM, and (b) a bisphosphonate active.

The present invention also relates to pharmaceutical compositions comprising a therapeutically effective amount of a geranylgeranyl disphosphate synthase inhibitor having an IC₅₀ value from about 0.01 nanoM to about 1000 nanoM.

The present invention also relates to pharmaceutical compositions comprising a therapeutically effective amount of the combination of: (a) a geranylgeranyl disphosphate synthase inhibitor having an IC₅₀ value from about 0.01 nanoM to about 1000 nanoM and (b) a bisphosphonate active.

The present invention also relates to the use of such compositions in the manufacture of a medicament for the methods disclosed herein.

The present invention also relates to the use of a composition in the manufacture of a medicament for treating or reducing the risk of contracting a disease state or condition involving bone tissue in a mammal comprising administering to a mammal in need thereof a therapeutically effective amount of a geranylgeranyl diphosphate synthase inhibitor having an IC₅₀ value from about 0.01 nanoM to about 100 0 nanoM.

The present invention also relates to the use of a composition in the manufacture of a medicament for treating or reducing the risk of contracting a disease state or condition involving bone tissue in a mammal comprising a therapeutically effective amount of a geranylgeranyl diphosphate synthase inhibitor having an IC₅₀ value from about 0.01 nanoM to about 100 0 nanoM.

All percentages and ratios used herein, unless otherwise indicated, are by weight. The invention hereof can comprise, consist of, or consist essentially of the essential as well as optional ingredients, components, and methods described herein.

### DETAILED DESCRIPTION OF THE INVENTION

The present invention relates to methods for identifying compounds useful as geranylgeranyl diphosphate synthase inhibitors and for inhibiting this enzyme with the compounds so identifited.

The mevalonate biosynthetic pathway is an important pathway of osteoclast function. This pathway is involved in the bisosynthesis of cholesterol and of isoprenoids, some of which are used in protein prenylation. It would be highly desirable to identify and develop compounds useful as selective inhibitors of geranylgeranyl diphosphate synthase in the osteoclasts. Such inhibitors would be useful for inhibiting ostetoclast function, thereby inhibiting undesired bone resorption and its manifestations in various disease states and conditions.

Geranylgeranyl diphosphate synthase is also known as GGPP synthases.

Alendronate (4-amino-1-hydroxybutylidene-1,1-bisphosphonate) is a potent inhibitor of bone resorption, used in the treatment and prevention of osteoporosis and other bone diseases. Without being limited by theory, it is believed that alendronate and other bisphosphonates are readily adsorbed onto the bone surface and are selectively taken up by osteoclasts during bone resorption. It is generally accepted that at the cellular level bisphosphonates act by inhibiting osteoclast activity. The effects of alendronate monosodium trihydrate and of the HMG-CoA reductase inhibitor, lovastatin, on osteoclasts in culture is known. Osteoclast formation and bone resorption are inhibited by alendronate monosodium trihydrate and by lovastatin. Mevalonic acid lactone or geranylgeraniol reverse the effects of lovastatin but only geranylgeraniol reverses the effects of alendronate, thereby supporting the hypothesis that alendronate monosodium trihydrate induces apoptosis by inhibiting protein prenylation via inhibition of the mevalonate pathway prior to the formation of geranylgeranyl diphosphate.

It is known that several nitrogen-containing bisphosphonates, including YM 175, EB 1053 and PHPBP, are potent, nanomolar inhibitors of rat liver squalene synthase. See, Amin D, Cornell S A, Gustafson S K, Needle S J, Ullrich J W, Bilder G E, and Perrone M H (1992) J. Lipid Res. 33: 1657-1663, which is incorporated by reference herein in its entirety. On the other hand, alendronate and pamidronate, two other nitrogen containing bisphosphonates, have comparatively little effect on squalene synthase. Alendronate and parnidronate, however, block sterol synthesis, as measured by the incorporation of ¹⁴C-MVA into sterol in a rat liver-cell free system, with respective IC₅₀'s of 168 nM and 420 nM, suggesting that these compounds inhibit another enzyme in the pathway. Without being limited by theory, it is therefore believed that nitrogen-containing bisphosphonates are potent inhibitors of any of several enzymes involved in isoprenoid synthesis.

The synthesis of geranylgeranyl diphosphate from mevalonate involves six enzymes, mevalonate (MVA) kinase (EC 2.7.1.36), phosphomevalonate (MVAP) kinase (EC 2.7.4.2), mevalonate diphosphate (MVAPP) decarboxylase, isopentenyl diphosphate (IPP) isomerase (EC 5.3.3.2), farnesyl diphosphate (FPP) synthase (EC 2.5.1.1), and geranylgeranyl diphosphate (GGPP) synthase. Farnesyl protein transferase (FTase), geranylgeranyl protein transferase I (GGTase I) and geranylgeranyl protein transferase II (GGTase II) are the enzymes responsible for prenylating proteins.

Methods of Identifying Inhibitors of Geranylgeranyl Diphosphate Synthase

The present invention relates to a method for identifying inhibitors of geranylgeranyl diphosphate synthase comprising:

a). contacting a putative geranylgeranyl diphosphate synthase inhibitor with a geranylgeranyl diphosphate synthase assay solution, and

b). determining, i.e. comparing, the geranylgeranyl diphosphate synthase activity of said assay solution with a geranylgeranyl diphosphate synthase assay solution not contacted with said putative inhibitor, in order to determine the amount of inhibition.

In these methods the geranylgeranyl diphosphatesynthase assay solution is typically an aqueous solution. The inhibition effect is measured with respect to the catalysis of an appropriate reaction that one of ordinary skill in the art can select. Reaction times, conditions, quatitation methods, and other variables are chosen for convenience to obtain a readily quantitated system for measuring the inhibition of the geranylgeranyl diphosphate synthase.

Additionally, in these methods of identifying inhibitors of geranylgeranyl diphosphate synthase, the enzyme can be used in a crude, unpurified state, from various tissues sources, e.g., liver. Alternatively, the enzyme can be used in a partially purified state, a purified state, or as an expressed form of the enzyme, e.g., the expressed human enzyme.

Methods of Inhibiting Bone Resorption

The present invention relates to methods for inhibiting bone resorption in a mammal comprising administering to a mammal in need thereof a therapeutically effective amount of a geranylgeranyl diphosphate synthase inhibitor.

The methods and compositions of the present invention are useful for both treating and reducing the risk of contracting disease states or conditions involving or associated with abnormal bone resorption. Such disease states or conditions include, but are not limited to, osteoporosis, glucocorticoid induced osteoporosis, Paget's disease, abnormally increased bone turnover, periodontal disease, arthritis, osteoarthritis, rheumatoid arthritis, tooth loss, bone fractures, rheumatoid arthritis, periprosthetic osteolysis, osteogenesis imperfecta, metastatic bone disease, hypercalcemia of malignancy, and multiple myeloma. The methods and compositions are also useful for both treating and reducing the risk of contracting other disease states or conditions mediated by geranylgeranyl disphosphate synthase.

In further embodiments, the methods comprise administering a therapeutically effective amount of the combination of (a) a geranylgeranyl diphosphate synthase inhibitor, which can itself be a bisphosphonate active, and (b) an additional bisphosphonate active. Both concurrent and sequential administration of the geranylgeranyl disphosphate synthase inhibitor and the additional bisphosphonate active are deemed within the scope of the present invention. With sequential administration, the geranylgeranyl diphosphate synthase inhibitor and the additional bisphosphonate can be administered in either order. In a subclass of sequential administration the geranylgeranyl diphosphate synthase inhibitor and the additional bisphosphonate are typically administered within the same 24 hour period. In yet a further subclass, the geranylgeranyl diphosphate synthase inhibitor and the additional bisphosphonate are typically administered within about 4 hours of each other.

The term “therapeutically effective amount”, as used herein, means that amount of the geranylgeranyl diphosphate synthase inhibitor, or other actives of the present invention, that will elicit the desired therapeutic effect or response or provide the desired benefit when administered in accordance with the desired treatment regimen. A preferred therapeutically effective amount is a bone resorption inhibiting amount.

“Pharmaceutically acceptable” as used herein, means generally suitable for administration to a mammal, including humans, from a toxicity or safety standpoint.

In the present invention, the geranylgeranyl diphosphate synthase inhibitor is typically administered for a sufficient period of time until the desired therapeutic effect is achieved. The term “until the desired therapeutic effect is achieved”, as used herein, means that the therapeutic agent or agents are continuously administered, according to the dosing schedule chosen, up to the time that the clinical or medical effect sought for the disease or condition being mediated is observed by the clinician or researcher. For methods of treatment of the present invention, the compounds are continuously administered until the desired change in bone mass or structure is observed. In such instances, achieving an increase in bone mass or a replacement of abnormal bone structure with normal bone structure are the desired objectives. For methods of reducing the risk of a disease state or condition, the compounds are continuously administered for as long as necessary to prevent the undesired condition. In such instances, maintenance of bone mass density is often the objective.

Nonlimiting examples of administration periods can range from about 2 weeks to the remaining lifespan of the mammal. For humans, administration periods can range from about 2 weeks to the remaining lifespan of the human, preferably from about 2 weeks to about 20 years, more preferably from about 1 month to about 20 years, more preferably from about 6 months to about 10 years, and most preferably from about 1 year to about 10 years.

Compositions of the Present Invention

The pharmaceutical compositions of the present invention comprise a therapeutically effective amount of a geranylgeranyl diphosphate synthase inhibitor.

These compositions can further comprise a pharmaceutically-acceptable carrier.

In further embodiments these compositions can also comprise an additional active.

Geranylgeranyl Diphosphate Synthase Inhibitor

The methods and compositions of the present invention comprise a geranylgeranyl diphosphate synthase inhibitor. These inhibitors can in themselves be bisphosphonates.

The geranylgeranyl diphosphate synthase inhibitors useful herein generally have an IC₅₀ value from about 0.01 nM to about 1000 nanoM, although inhibitors with activities outside this range can be useful depending upon the dosage and route of administration. In a subclass of the present invention, the inhibitors have an IC₅₀ value of from about 0.01 nM to about 100 nM. In a further subclass of the present invention, the inhibitors have an IC₅₀ value of from about 0.01 nM to about 1 nM. IC₅₀ is a common measure of inhibition activity well known to those of ordinary skill in the art and is defined as the concentration of the inhibitor needed to obtain a 50% reduction in the activity of the geranylgeranyl disphosphate synthase.

The combination of two or more gem aylgeranyl diphosphate synthase inhibitors are also deemed as within the scope of the present invention.

The precise dosage of the geranylgeranyl diphosphate synthase inhibitor will vary with the dosing schedule, the particular compound chosen, the age, size, sex and condition of the mammal or human, the nature and severity of the disorder to be treated, and other relevant medical and physical factors. Thus, a precise pharmaceutically effective amount cannot be specified in advance and can be readily determined by the caregiver or clinician. Appropriate amounts can be determined by routine experimentation from animal models and human clinical studies. Generally, an appropriate amount is chosen to obtain an inhibition of the geranylgeranyl diphosphate synthase activity so as to obtain a bone resorption inhibiting effect.

For humans, an effective oral dose of the geranylgeranyl diphosphate synthase inhibitor is about 1 μg/kg to about 1000 μg/kg, preferably about 10 μg/kg, for a human subject.

For the geranylgeranyl diphosphate synthase inhibitor, human doses which can be administered are generally in the range of about 0.1 mg/day to about 10 mg/day, preferably from about 0.25 mg/day to about 5 mg/day, and more preferably from about 0.5 mg/day to about 1.5 mg/day, based on an active weight basis. A typical nonlimiting dosage amount would be about 0.75 mg/day. The pharmaceutical compositions herein comprise from about 0.1 mg to about 10 mg, preferably from about 0.25 mg to about 5 mg, and more preferably from about 0.5 mg to about 1.5 mg of the geranylgeranyl diphosphate synthase inhibitor. A typical nonlimiting amount is about 0.75 mg.

Bisphosphonates

The methods and compositions of the present invention, can further comprise a bisphosphonate active or a pharmaceutically acceptable salt thereof. These bisphosphonate actives are defined herein to be distinct from and not to included the geranylgeranyl diphosphate synthase inhibitors of the present invention, because certain nitrogen-containing bisphosphonates, e.g., alendronate are found to have activity as geranylgeranyl diphosphate synthase inhibitors. In other words, the present invention can include the combination of a geranylgeranyl diphosphate synthase inhibitor which happens to have a bisphosphonate structure and an additional bisphosphonate active which does not necessarily have activity as a geranylgeranyl diphosphate synthase inhibitor.

The term “nitrogen-containing” as used herein means that the bisphosphonate compound or pharmaceutically acceptable salt thereof comprises at least one nitrogen atom in the bisphosphonate portion of the molecule. In other words, for a pharmaceutically-acceptable salt of the bisphosphonate, any nitrogen atom contained in the positive counter ion of such a salt, e.g., the nitrogen atom of an ammonium counter ion, would not be considered in meeting the “nitrogen-containing” definition. For example, alendronic acid, i.e. 4-amino-1-hydroxybutylidene-1,1-bisphosphonic acid is an example of a nitrogen-containing bisphosphonate. However, the ammonium salt of the unsubstituted 1-hydroxybutylidene-1,1-bisphosphonic acid would not be a nitrogen-containing bisphosphonate as defined herein.

In certain embodiments, the methods and compositions of the present invention comprise a bisphosphonate. The bisphosphonates of the present invention correspond to the chemical formula

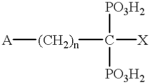

wherein n is an integer from 0 to about 7 and wherein A and X are independently selected from the group consisting of H, OH, halogen, NH₂, SH, phenyl, C1-C30 alkyl, C3-C30 branched or cycloalkyl, C1-C30 substituted alkyl, C1-C10 alkyl substituted NH₂, C3-C10 branched or cycloalkyl substituted NH₂, C1-C10 dialkyl substituted NH₂, C3-C10 branched or cycloalkyl disubstituted NH₂, C1-C10 alkoxy, C1-C10 alkyl substituted thio, thiophenyl, halophenylthio, C1-10 alkyl substituted phenyl, pyridyl, furanyl, pyrrolidinyl, imidazolyl, imidazopyridinyl, and benzyl, such that both A and X are not selected from H or OH when n is 0; or A and X are taken together with the carbon atom or atoms to which they are attached to form a C3-C10 ring.

In the foregoing chemical formula, the alkyl groups can be straight, branched, or cyclic, provided that sufficient atoms are selected for the chemical formula. The C1-C30 substituted alkyl can include a wide variety of substituents, nonlimiting examples which include those selected from the group consisting of phenyl, pyridyl, furanyl, pyrrolidinyl, imidazonyl, NH₂, C1-C10 alkyl or dialkyl substituted NH₂, OH, SH, and C1-C10 alkoxy.

The foregoing chemical formula is also intended to encompass complex carbocyclic, aromatic and hetero atom structures for the A and/or X substituents, nonlimiting examples of which include naphthyl, quinolyl, isoquinolyl, adamantyl, and chlorophenylthio.

A non-limiting class of structures useful in the instant invention are those in which A is selected from the group consisting of H, OH, and halogen, X is selected from the group consisting of C1-C30 alkyl, C1-C30 substituted alkyl, halogen, and C1-C10 alkyl or phenyl substituted thio, and n is 0.

A non-limiting subclass of structures useful in the instant invention are those in which A is selected from the group consisting of H, OH, and Cl, X is selected from the group consisting of C1-C30 alkyl, C1-C30 substituted alkyl, Cl, and chlorophenylthio, and n is 0.

A non-limiting example of the subclass of structures useful in the instant invention is when A is OH and X is a 3-aminopropyl moiety, and n is 0, so that the resulting compound is a 4-amino-1,-hydroxybutylidene-1,1-bisphosphonate, i.e. alendronate.

Pharmaceutically acceptable salts and derivatives of the bisphosphonates are also useful herein. Nonlimiting examples of salts include those selected from the group consisting alkali metal, alkaline metal, ammonium, and mono-, di, tri-, or tetra-C1-C30-alkyl-substituted ammonium. Preferred salts are those selected from the group consisting of sodium, potassium, calcium, magnesium, and ammonium salts. More preferred are sodium salts including mono and di and other higher sodium salts. Nonlimiting examples of derivatives include those selected from the group consisting of esters, hydrates, and arnides. Hydrates can include whole number hydrates, i.e. monohydrates, dihydrates, trihydrates, etc., as well as fractional hydrates, such as for example, a hemi-pentahydrate (i.e. a 2.5 hydrate). Anhydrous forms of the bisphosphonates are also contemplated as within the scope of the present invention.

“Pharmaceutically acceptable” as used herein means that the salts and derivatives of the bisphosphonates have the same general pharmacological properties as the free acid form from which they are derived and are acceptable from a toxicity viewpoint.

It should be noted that the terms “bisphosphonate” and “bisphosphonates”, as used herein in referring to the therapeutic agents of the present invention are meant to also encompass diphosphonates, biphosphonic acids, and diphosphonic acids, as well as salts and derivatives of these materials. The use of a specific nomenclature in referring to the bisphosphonate or bisphosphonates is not meant to limit the scope of the present invention, unless specifically indicated. Because of the mixed nomenclature currently in use by those or ordinary skill in the art, reference to a specific weight or percentage of a bisphosphonate compound in the present invention is on an acid active weight basis, unless indicated otherwise herein. For example, the phrase “about 70 mg of a bone resorption inhibiting bisphosphonate selected from the group consisting of alendronate, pharmaceutically acceptable salts thereof, and mixtures thereof, on an alendronic acid active weight basis” means that the amount of the bisphosphonate compound selected is calculated based on 70 mg of alendronic acid.

Nonlimiting examples of bisphosphonates useful herein include the following:

Alendronic acid, 4-amino-1-hydroxybutylidene-1,1-bisphosphonic acid.

Alendronate (also known as alendronate sodium or monosodium trihydrate), 4-amino-1-hydroxybutylidene-1,1-bisphosphonic acid monosodium trihydrate.

Alendronic acid and alendronate are described in U.S. Pat. No. 4,922,007, to Kieczykowski et al., issued May 1, 1990, and U.S. Pat. No. 5,019,651, to Kieczykowski, issued May 28, 1991, both of which are incorporated by reference herein in their entirety.

1,1-dichloromethylene-1,1-diphosphonic acid (clodronic acid), and the disodium salt (clodronate, Procter and Gamble), are described in Belgium U.S. Pat. No. 672,205 (1966) and J. Org. Chem 32, 4111 (1967), both of which are incorporated by reference herein in their entirety.

1-hydroxy-3-(1-pyrrolidinyl)-propylidene-1,1-bisphosphonic acid (EB-1053).

1-hydroxyethane-1,1-diphosphonic acid (etidronic acid).

1-hydroxy-3-(N-methyl-N-pentylamino)propylidene-1,1-bisphosphonic acid, also known as BM-210955, Boehringer-Mannheim (ibandronate), is described in U.S. Pat. No. 4,927,814, issued May 22, 1990, which is incorporated by reference herein in its entirety.

Cycloheptylaminomethylene-1,1-bisphosphonic acid, YM 175, Yamanouchi (incadronate, formerly known as cimadronate), as described in U.S. Pat. No. 4,970,335, to Isomura et al., issued Nov. 13, 1990, which is incorporated by reference herein in its entirety.

1-hydroxy-2-imidazo-(1,2-a)pyridin-3-yethylidene (minodronate).

6-amino-1-hydroxyhexylidene-1,1-bisphosphonic acid (neridronate).

3-(dimethylamino)-1-hydroxypropylidene-1,1-bisphosphonic acid (olpadronate).

3-amino-1-hydroxypropylidene-1,1-bisphosphonic acid (pamidronate).

[2-(2-pyridinyl)ethylidene]-1,1-bisphosphonic acid (piridronate) is described in U.S. Pat. No. 4,761,406, which is incorporated by reference in its entirety.

1-hydroxy-2-(3-pyridinyl)-ethylidene-1,1-bisphosphonic acid (risedronate).

(4-chlorophenyl)thiomethane-1,1-disphosphonic acid (tiludronate) as described in U.S. Pat. No. 4,876,248, to Breliere et al., Oct. 24, 1989, which is incorporated by reference herein in its entirety.

1-hydroxy-2-(lH-imidazol-1-yl)ethylidene-1,1-bisphosphonic acid (zolendronate).

Preferred are bisphosphonates selected from the group consisting of alendronate, clodronate, etidronate, ibandronate, incadronate, minodronate, neridronate, risedronate, piridronate, pamidronate, tiludronate, zoledronate, pharmaceutically acceptable salts or esters thereof, and mixtures thereof.

More preferred is alendronate, ibandronate, risedronate, pharmaceutically acceptable salts or esters thereof, and mixtures thereof.

More preferred is alendronate, pharmaceutically acceptable salts thereof, and mixtures thereof.

Most preferred is alendronate monosodium trihydrate.

In other embodiments, other preferred salts are the sodium salt of ibandronate, and risedronate monosodium hemi-pentahydrate (i.e. the 2.5 hydrate of the monosodium salt).

It is recognized that mixtures of two or more of the bisphosphonate actives can be utilized.

The precise dosage of the bisphosphonate will vary with the dosing schedule, the particular bisphosphonate chosen, the age, size, sex and condition of the mammal or human, the nature and severity of the disorder to be treated, and other relevant medical and physical factors. Thus, a precise therapeutically effective amount cannot be specified in advance and can be readily determined by the caregiver or clinician. Appropriate amounts can be determined by routine experimentation from animal models and human clinical studies. Generally, an appropriate amount of bisphosphonate is chosen to obtain a bone resorption inhibiting effect, i.e. a bone resorption inhibiting amount of the nitrogen-containing bisphosphonate is administered. For humans, an effective oral dose of nitrogen-containing bisphosphonate is typically from about 1.5 to about 6000 μg/kg body weight and preferably about 10 to about 2000 μg/kg of body weight.

For the bisphosphonate, alendronate monosodium trihydrate, common human doses which are administered are generally in the range of about 2 mg/day to about 40 mg/day, preferably about 5 mg/day to about 40 mg/day. In the U.S. presently approved dosages for alendronate monosodium trihydrate are 5 mg/day for preventing osteoporosis, 10 mg/day for treating osteoporosis, and 40 mg/day for treating Paget's disease.

In alternative dosing regimens, the bisphosphonate can be administered at intervals other than daily, for example once-weekly dosing, twice-weekly dosing, biweekly dosing, and twice-monthly dosing. In such dosing regimens, appropriate multiples of the bisphosphonate dosage would be administered. For example, in a once weekly dosing regimen, alendronate monosodium trihydrate would be administered at dosages of 35 mg/week or 70 mg/week in lieu of seven consecutive daily dosages of 5 mg or 10 mg.

For ibandronate the unit dosage can comprises from about 2.5 mg to about 200 mg, on an ibandronic acid active weight basis, i.e. calculated on the basis of the corresponding acid. Examples of daily oral dosages comprise about 2.5 mg, 3.5 mg, 5 mg, 7.5 mg, and 10 mg. Examples of weekly oral dosages include a unit dosage which is useful for inhibiting bone resorption, and treating and preventing osteoporosis selected from the group consisting of about 25 mg, 30 mg, 35 mg, 40 mg, 45 mg, or 50 mg.

For risedronate the unit dosage can comprise from about 2.5 mg to about 200 mg, on a risedronic acid active weight basis, i.e. calculated on the basis of the corresponding acid. Examples of daily oral dosages comprise about 2.5 mg, 3.5 mg, 5 mg (an exemplary osteoporosis daily dosage), 7.5 mg, and 10 mg, and 30 mg (an exemplary Paget's disease daily dosage). Examples of weekly oral dosages include a unit dosage which is useful for inhibiting bone resorption, and treating and preventing osteoporosis selected from the group consisting of about 25 mg, 30 mg, 35 mg, 40 mg, 45 mg, or 50 mg.

The pharmaceutical compositions herein comprise from about 1 mg to about 100 mg of bisphosphonate, preferably from about 2 mg to 70 mg, and more preferably from about 5 mg to about 70, on a bisphosphonic acid basis. For the bisphosphonate alendronate monosodium trihydrate, the pharmaceutical compositions useful herein comprise about 2.5 mg, 5 mg, 10 mg, 35, mg, 40 mg, or 70 mg of the active on an alendronic acid active weight basis.

See also, U.S. Pat. No. 4,610,077, to Rosini et al., issued Nov. 4, 1986; U.S. Pat. No. 5,358,941, to Bechard et al., issued Oct. 25, 1994; and PCT application number WO 99/04773, to Daifotis et al., published Feb. 4, 1999; all of which are incorporated by reference herein in their entirety.

Other Bone Agents

Further embodiments of the methods and compositions of the present invention can comprise additional bone agents useful for inhibiting bone resorption and providing the desired therapeutic benefits of the invention. Examples of such agents include those selected from the group consisting of calcitonin, estrogens,progesterone, androgens, calcium supplements, fluoride, growth hormone secretagogues, vitamin D analogues, and selective estrogen receptor modulators. The calcitonins useful herein can be from human or nonhuman sources, e.g. salmon calcitonin. Nonlimiting examples of estrogens include estradiol. Nonlimiting examples of selective estrogen receptor modulators include raloxifene, iodoxifene, and tamoxifene. Growth horomone secretagogues are described in U.S. Pat. No. 5,536,716, to Chen et al., issued Jul. 16, 1996, which is incorporated by reference herein in its entirety.

Other Components of the Pharmaceutical Compositions

The geranylgeranyl diphosphate synthase inhibitors, and in further embodiments the bisphosphonate actives and any other additional actives, are typically administered in admixture with suitable pharmaceutically acceptable diluents, excipients, or carriers, collectively referred to herein as “carrier materials”, suitably selected with respect to the mode of administration. Nonlimiting examples of product forms include tablets, capsules, elixirs, syrups, powders, suppositories, nasal sprays, liquids for ocular administration, formulations for transdermal administration, and the like, consistent with conventional pharmaceutical practices. For example, for oral administration in the form of a tablet, capsule, or powder, the active ingredient can be combined with an oral, non-toxic, pharmaceutically acceptable inert carrier such as lactose, starch, sucrose, glucose, methyl cellulose, magnesium stearate, mannitol, sorbitol, croscarmellose sodium and the like. For oral administration in liquid form, e.g., elixirs and syrups, the oral drug components are combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated. Suitable binders can include starch, gelatin, natural sugars such a glucose, anhydrous lactose, free-flow lactose, beta-lactose, and corn sweeteners, natural and synthetic gums, such as acacia, guar, tragacanth or sodium alginate, carboxymethyl cellulose, polyethylene glycol, waxes, and the like. Lubricants used in these dosage forms include sodium oleate, sodium stearate, magnesium stearate, sodium benzoate, sodium acetate, sodium chloride and the like. An example of a tablet formulation is that described in U.S. Pat. No. 5,358,941, to Bechard et al, issued Oct. 25, 1994, which is incorporated by reference herein in its entirety. The compounds used in the present method can also be coupled with soluble polymers as targetable drug carriers. Such polymers can include polyvinylpyrrolidone, pyran copolymer, polyhydroxylpropyl-methacrylamide, and the like.

The following Examples are presented to better illustrate the invention.

### EXAMPLE 1

Pharmaceutical tablets: the tablets are prepared using standard mixing and formation techniques.

Tablets containing about 1 to 100 mg of a geranylgeranyl diphosphate synthase inhibitor are prepared using the following relative weights of ingredients.

| | |
|---|---|
| Ingredient | Per Tablet |

| | | |
|---|---|---|
| Geranylgeranyl Diphosphate Synthase Inhibitor | 0.10 to 10 | mg |
| Anhydrous Lactose, NF | 71.32 | mg |
| Magnesium Stearate, NF | 1.0 | mg |
| Croscarmellose Sodium, NF | 2.0 | mg |
| Microcrystalline Cellulose, NF | QS 200 | mg |

In further embodiments, tablets are prepared that also contain 5 or 10 mg of a bisphosphonate active, on a bisphosphonic acid active basis, of a bisphosphonate selected from the group consisting of alendronate cimadronate, clodronate, tiludronate, etidronate, ibandronate, neridronate, olpandronate, risedronate, piridronate, pamidronate, zoledronate, and pharmaceutically acceptable salts thereof.

### EXAMPLE 2

Liquid formulation: liquid formulations are prepared using standard mixing techniques.

A liquid formulation containing about 1 to about 100 mg of a geranylgeranyl diphosphate synthase inhibitor is prepared using the following relative weights of ingredients.

| | |
|---|---|
| Ingredient | Weight |

| | | |
|---|---|---|
| Geranylgeranyl Diphosphate Synthase Inhibitor | 0.10 to 10 | mg |
| Sodium Propylparaben | 22.5 | mg |
| Sodium Butylparaben | 7.5 | mg |
| Sodium Citrate Dihydrate | 1500 | mg |
| Citric Acid Anhydrous | 56.25 | mg |
| Sodium Saccharin | 7.5 | mg |
| Water | qs 75 | mL |

| | |
|---|---|
| 1 N Sodium Hydroxide (aq) | qs pH 6.75 |

The resulting liquid formulation is useful for administration for inhibiting bone resorption.

In further embodiments solutions are prepared also containing 5 or 10 mg of a bisphosphonate active, on a bisphosphonic acid active basis, of a bisphosphonate selected from the group consisting of alendronate cimadronate, clodronate, tiludronate, etidronate, ibandronate, neridronate, olpandronate, risedronate, piridronate, pamidronate, zoledronate, and pharmaceutically acceptable salts thereof.

What is claimed is:

1. A method for identifying an inhibitor of geranylgeranyl diphosphate synthase comprising:
a). contacting a putative geranylgeranyl diphosphate synthase inhibitor with a geranylgeranyl diphosphate synthase solution, and
b). determining the geranylgeranyl diphosphate synthase activity of said solution with a geranylgeranyl diphosphate synthase solution not contacted with said putative inhibitor.

2. A method according to claim 1 wherein said geranylgeranyl diphosphate synthase is an expressed human geranylgeranyl diphosphate synthase protein.

3. A method for inhibiting geranylgeranyl diphosphate synthase activity in a mammal comprising administering to a mammal in need thereof a therapeutically effective amount of a geranylgeranyl diphosphate synthase inhibitor having an IC₅₀ value from about 0.01 nanoM to about 100 0 nanoM.

4. A method according to claim 3 wherein said mammal is a human.

5. A method for treating or reducing the risk of contracting a disease state or condition involving bone tissue in a mammal comprising administering to a mammal in need thereof a therapeutically effective amount of a geranylgeranyl diphosphate synthase inhibitor having an IC₅₀ value from about 0.01 nanoM to about 100 0 nanoM.

6. A method according to claim 5 wherein said mammal is a human.

7. A method according to claim 6 wherein said disease state or condition is selected from the group consisting of osteoporosis, glucocorticoid induced osteoporosis, Paget's disease, abnormally increased bone turnover, periodontal disease, arthritis, osteoarthritis, rheumatoid arthritis, tooth loss, bone fractures, rheumatoid arthritis, periprosthetic osteolysis, osteogenesis imperfecta, metastatic bone disease, hypercalcemia of malignancy, and multiple myeloma.

8. A method according to claim 7 wherein said disease state or condition is selected from the group consisting of osteoporosis, glucocorticoid induced osteroporosis, and Paget's disease.

9. A method for inhibiting geranylgeranyl diphosphate synthase activity in a mammal comprising administering to a mammal in need thereof a therapeutically effective amount of the combination of:
(a) a geranylgeranyl diphosphate synthase inhibitor having an IC₅₀ value from about 0.01 nanoM to about 1000 nanoM, and
(b) a bisphosphonate active.

10. A method according to claim 9 wherein said mammal is a human.

11. A method for inhibiting bone resorption in a mammal comprising administering to a mammal in need thereof a therapeutically effective amount of the combination of:
(a) a geranylgeranyl diphosphate synthase inhibitor having an IC₅₀ value from about 0.01 nanoM to about 1000 nanoM, and
(b) a bisphosphonate active.

12. A method according to claim 11 wherein said mammal is a human.

13. A method for treating or reducing the risk of contracting a disease state or condition involving bone tissue in a mammal comprising administering to a mammal in need thereof a therapeutically effective amount of the combination of:
(a) a geranylgeranyl diphosphate synthase inhibitor having an IC₅₀ value from about 0.01 nanoM to about 1000 nanoM, and
(b) a bisphosphonate active.

14. A method according to claim 13 wherein said mammal is a human.

15. A method according to claim 14 wherein said disease state or condition is selected from the group consisting of osteoporosis, glucocorticoid induced osteoporosis, Paget's disease, abnormally increased bone turnover, periodontal disease, arthritis, osteoarthritis, rheumatoid arthritis, tooth loss, bone fractures, rheumatoid arthritis, periprosthetic osteolysis, osteogenesis imperfecta, metastatic bone disease, hypercalcemia of malignancy, and multiple myeloma.

16. A method according to claim 15 wherein said disease state or condition is selected from the group consisting of osteoporosis, glucocorticoid induced osteoporosis, and Paget's disease.

17. A method according to claim 16 wherein said bisphosphonate active corresponds to the chemical structure 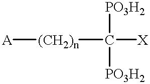
wherein n is an integer from 0 to 7 and wherein A and X are independently selected from the group consisting of H, OH, halogen, NH₂, SH, phenyl, C1-C30 alkyl, C3-C30 branched or cycloalkyl, C1-C30 substituted alkyl, C1-C10 alkyl substituted NH₂, C3-C10 branched or cycloalkyl substituted NH₂, C1-C10 dialkyl substituted NH₂, C1-C10 alkoxy, C1-C10 alkyl substituted thio, thiophenyl, halophenylthio, C1-C10 alkyl substituted phenyl, pyridyl, furanyl, pyrrolidinyl, imidazolyl, imidazopyridinyl, and benzyl; or A and X are taken together with the carbon atom or atoms to which they are attached to form a C3-C10 ring; and provided that when n is 0, A and X are not selected from the group consisting of H and OH; and the pharmaceutically acceptable salts thereof.

18. A method according to claim 17 wherein said bisphosphonate active is selected from the group consisting of alendronate, cimadronate, clodronate, tiludronate, etidronate, ibandronate, neridronate, olpandronate, risedronate, piridronate, pamidronate, zoledronate, pharmaceutically acceptable salts thereof, and mixtures thereof.

19. A method according to claim 18 wherein said bisphosphonate active is alendronate, pharmaceutically acceptable salts thereof, and mixtures thereof.

20. A method according to claim 19 wherein said bisphosphonate active is alendronate monosodium trihydrate.

21. A pharmaceutical composition comprising a therapeutically effective amount of a geranylgeranyl diphosphate synthase inhibitor having an IC₅₀ value from about 0.01 nanoM to about 1000 nanoM.

22. A pharmaceutical composition comprisig a therapeutically effective amount of the combination of:
(a) a geranylgeranyl diphosphate synthase inhibitor having an IC₅₀ value from about 0.01 nanoM to about 1000 nanoM, and
(b) a bisphosphonate active.

23. A pharmaceutical composition according to claim 22 wherein said bisphosphonate active corresponds to the chemical structure 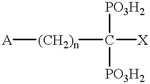
wherein n is an integer from 0 to 7 and wherein A and X are independently selected from the group consisting of H, OH, halogen, NH₂, SH, phenyl, C1-C30 alkyl, C3-C30 branched or cycloalkyl, C1-C30 substituted alkyl, C1-C10 alkyl substituted NH₂, C3-C10 branched or cycloalkyl substituted NH₂, C1-C10 dialkyl substituted NH₂, C1-C10 alkoxy, C1-C10 alkyl substituted thio, thiophenyl, halophenylthio, C1-C10 alkyl substituted phenyl, pyridyl, furanyl, pyrrolidinyl, imidazolyl, imidazopyridinyl, and benzyl; or A and X are taken together with the carbon atom or atoms to which they are attached to form a C3-C10 ring; and provided that when n is 0, A and X are not selected from the group consisting of H and OH; and the pharmaceutically acceptable salts thereof.

24. A pharmaceutical composition according to claim 23 wherein said bisphosphonate active is selected from the group consisting of alendronate, cimadronate, clodronate, tiludronate, etidronate, ibandronate, neridronate, olpandronate, risedronate, piridronate, pamidronate, zoledronate, pharmaceutically acceptable salts thereof, and mixtures thereof.

25. A pharmaceutical composition according to claim 24 wherein said bisphosphonate active is alendronate, pharmaceutically acceptable salts thereof, and mixtures thereof.

26. A pharmaceutical composition according to claim 25 wherein said bisphosphonate active is alendronate monosodium trihydrate.

27. The use of a composition in the manufacture of a medicament for treating or reducing the risk of contracting a disease state or condition involving bone tissue in a mammal comprising administering to a mammal in need thereof a therapeutically effective amount of a geranylgeranyl diphosphate synthase inhibitor having an IC₅₀ value from about 0.01 nanoM to about 1000 nanoM.

28. The use of a composition in the manufacture of a medicament for treating or reducing the risk of contracting a disease state or condition involving bone tissue in a mammal comprising a therapeutically effective amount of a geranylgeranyl diphosphate synthase inhibitor having an IC₅₀ value from about 0.01 nanoM to about 1000 nanoM.
